(19) United States
(12) Patent Application Publication
(10) Pub. No.: US 2014/0024960 A1
(43) Pub. Date: Jan. 23, 2014

(54) SENSOR DEVICE AND MOUTHPIECE

(71) Applicants: Trevor Smith, Maidstone (GB); Jason Smith, Gillingham (GB); Ron Logan-Sinclair, Rochester (GB)

(72) Inventors: Trevor Smith, Maidstone (GB); Jason Smith, Gillingham (GB); Ron Logan-Sinclair, Rochester (GB)

(21) Appl. No.: 13/944,005

(22) Filed: Jul. 17, 2013

(30) Foreign Application Priority Data
Jul. 18, 2012 (GB) GB1212736.1
Dec. 11, 2012 (GB) GB1222281.6

(51) Int. Cl. A61B 5/097; A61B 5/08

(52) U.S. Cl. CPC A61B 5/097; A61B 5/082
USPC 600/532; 600/543

(57) ABSTRACT

The present invention relates to gas sensing devices and to mouthpieces therefor. More particularly it relates to gas sensor devices for detecting gases in exhaled air, and to improved mouthpieces and mouthpiece interfaces for use with such devices. The invention also provides methods for using such devices and kits.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### FIELD OF THE INVENTION

The present invention relates to gas sensing devices and to mouthpieces therefor. More particularly it relates to gas sensor devices for detecting gases in exhaled air, and to improved mouthpieces and interfaces for use with such devices. The invention also provides methods for using such devices and kits.

### BACKGROUND OF THE INVENTION

Breath analysis, i.e. the analysis of the components of exhaled air, is emerging as a powerful diagnostic and prognostic tool in healthcare. For example, the detection of exhaled nitric oxide (ENO) is used for the identification of inflammation in the lungs and airways, e.g. for the diagnosis of asthma and other inflammatory conditions. Detecting hydrogen in breath can be used to detect or monitor lactose intolerance and carbohydrate maladsorption. Ammonia levels can be determined to investigate H. pylori activity in the stomach.

There are now a substantial number of devices available for conducting breath analysis. Much innovation has occurred in the area of sensor technology to allow faster and more accurate detection of target gases, and there has also been development in the clinical methodology techniques used in taking samples.

There has also been some development in respect of mouthpieces for ENO sampling, where it is important that a back pressure is applied and that the exhalation rate is maintained within specific boundaries. However, the mouthpieces used in the other areas of breath analysis, where carefully controlled rates of exhalation and application of back pressure is not of paramount importance, have not been developed.

For typical breath detectors, such as carbon monoxide and hydrogen detectors, mouthpieces comprise relatively wide-bore (i.e. 22 mm or higher) cardboard tubes which typically have a circular or elliptical cross section. These mouthpieces are disposable, typically with a new mouthpiece being used for each subject. These are essentially the same mouthpieces used for spirometry devices, and indeed spirometry mouthpieces were simply adopted for breath analysis devices when they began to emerge. There has been no move to improve these mouthpieces as they have been considered to be the perfectly optimal solution. The present inventors have, however, identified significant drawbacks associated with the use of such mouthpieces. For example, although light in weight, conventional mouthpieces are bulky and take up a significant volume when packaged—this has implications in terms of shipping and storage of mouthpieces, particularly as they are often required in large numbers. The wide nature of the mouthpieces can cause problems in that a subject has difficulty in controlling their exhalation rate through the mouthpiece; the exhalation rate subject through the mouthpiece can therefore be too rapid, meaning that the user runs out of breath before a sample is taken or that samples are taken incorrectly, e.g. due to the sensor not having enough sample to analyse.

### BRIEF SUMMARY OF THE INVENTION

The present invention provides improved mouthpiece systems for use in breath analysis.

In a first aspect the present invention provides an interface for the attachment of a mouthpiece to a gas sensor device, the interface comprising a first mouthpiece engaging means for engaging a first type of mouthpiece, and a second mouthpiece engaging means for engaging a second type of mouthpiece, the interface comprising an inlet positioned such that in use it is in fluid communication with the lumen of a mouthpiece mounted on either the first or the second mouthpiece engaging means.

Suitably the first mouthpiece engaging means is adapted to engage a larger mouthpiece than the second mouthpiece engaging means. This allows wide-bore mouthpieces (e.g. conventional mouthpieces) or narrow-bore mouthpieces to be connected to the same breath sensor device.

In preferred embodiment the first and/or second mouthpiece engaging means suitably comprises a projection adapted to engage with the mouthpiece. The projection is preferably a flange. Conveniently both the first and second mouthpiece engaging means comprise flanges. A mouthpiece can be mounted over a suitably shaped flange, e.g. a tubular mouthpiece can be pushed over the flange. Alternatively, a mouthpiece can be mounted inside a flange, i.e. within a lumen defined by the flange. The flange can be continuous or discontinuous, provided that it provides sufficient support to mount a mouthpiece thereon.

Alternatively, the first and/or second mouthpiece engaging means can comprise a recess, e.g. a groove, adapted to engage with the mouthpiece. A mouthpiece can be mounted by inserting it into a suitably shaped recess.

In one particularly preferred embodiment the first and second mouthpiece engaging means are defined by a flange, the outside surface of which defines the first mouthpiece engaging means and the inner surface of which defines the second mouthpiece engaging means. Thus, the first mouthpiece engaging means is provided by a projection, i.e. the flange, and the second mouthpiece engaging means is defined by a recess, i.e. the lumen of said flange. Such an embodiment provides a significant advantage in that no trough is present between two flanges, and thus avoids the risk of condensation or the like being forming or being retained in the trough, as discussed further below.

It is preferred that the first and/or second mouthpiece engaging means are tapered such that a mouthpiece engages more tightly as it is pushed further onto or into the engaging means. For example, where the mouthpiece engaging means is a flange over which a mouthpiece is mounted, the flange preferably tapers outwards in the proximal direction (i.e. towards the sensor device) and thus a mouthpiece engages more tightly with the inside of the mouthpiece as it is pushed further onto the engaging means. Where the mouthpiece engaging means is a recess into which the mouthpiece is mounted (e.g. the lumen defined by a flange), it is preferred that the recess tapers inwardly so it engages more tightly with the outside of a mouthpiece as it is pushed further into the engaging means.

The first and/or second mouthpiece engaging means can suitably comprise a shoulder adapted to abut against the leading edge of a mouthpiece when it is located in the correct position on the mouthpiece engaging means.

Preferably the first and/or second mouthpiece engaging means define a circle, ellipse, oval or polygon. It will be apparent that a range of mouthpiece shapes could be used, provided that they allow a user to form a relatively airtight seal with their lips. In a preferred embodiment the first mouthpiece engaging means defines an ellipse and the second mouthpiece engaging means defines a circle. Thus a particularly preferred embodiment comprises an elliptical flange for mounting a wide-bore mouthpiece and a circular flange or recess for mounting a narrow-bore mouthpiece.

It should be noted that in general the various terms for shapes are not used in their strict geometric sense and are intended to encompass shapes which deviate to some extent from strict mathematical rules. For example, the term ‘ellipse’ is intended to cover true ellipses but also other generally flattened circles or rounded lozenge shapes.

Suitably the second mouthpiece engaging means is located within the ambit of the first mouthpiece engaging means. The first and second mouthpiece engaging means can be concentric, for example.

The second mouthpiece engaging means is preferably adapted to engage a mouthpiece having a lumen with a cross-sectional area of 180 mm² or less, more preferably 80 mm² or less, optionally 50 mm² or less. These areas correlate with circular tubes of approximate diameter 15 mm, 10 mm and 8 mm, respectively. However, it should be noted that the mouthpiece need not be circular, but could also be elliptical, oval or polygonal, as discussed above. For non-circular cross-section mouthpieces, one can think in terms of mouthpieces having a lumen with a major width dimension of 15 mm or less, preferably 10 mm or less. By ‘major width dimension’ is meant the width of the lumen of the mouthpiece when measured in its widest dimension, e.g. the major axis of an ellipse. Furthermore it should be noted that the mouthpiece can have a constant cross section along its length, or it could have a cross-section that varies along its length in terms of shape or area. The relevant measurement is typically taken at the narrowest point along the length of the mouthpiece, as it is the narrowest point which will dictate the resistance to flow of exhaled air along the mouthpiece. Such mouthpieces will be referred to at points below as ‘narrow-bore’ mouthpieces.

The first mouthpiece engaging means is suitably adapted to engage a mouthpiece having a lumen with a cross-sectional area of 300 mm² or more, more preferably 380 mm² or more, optionally 700 mm² or more. These areas correlate with circular tubes of approximate diameter 20 mm, 22 mm and 30 mm, respectively. The above discussion with respect to the various shapes of cross-sections of the second mouthpiece applies in the same way in respect of the first mouthpiece. Such mouthpieces will be referred to at points below as ‘wide-bore’ mouthpieces.

Preferably the inlet through which the exhaled air passes into the breath sensor device is positioned within the ambit of the second mouthpiece engaging means. Typically the inlet has a similar area to the cross-sectional area of the mouthpiece, although it could be narrower.

In a second aspect the present invention provides a gas sensor device comprising an interface as set out above. In a preferred embodiment the gas sensor device is a CO or H₂ sensor device. However, it could be any other type of gas sensor device which is used to measure a property of exhaled air.

The interface may comprise fixing means to fix the interface to a gas sensor device. For example the fixing means could comprise clip elements, holes for screws to engage with, or the like. The interface may alternatively be integral with a gas sensor device, e.g. formed as part of the casing of the device.

In a further aspect the present invention also provides a gas sensor device comprising a mouthpiece attached to the first or second mouthpiece engaging means. In a preferred embodiment there is provided such a gas sensor device comprising a mouthpiece having a cross-sectional area of 180 mm² or less, more preferably 80 mm² or less, optionally 50 mm² or less. Preferably the mouthpiece has a substantially circular cross-section and has a diameter of 10 mm or less attached to the second mouthpiece engaging means. Suitably the mouthpiece is from 70 mm to 130 mm long, preferably from 80 mm to 120 mm long, and more preferably from 90 mm to 110 mm long. Suitably the mouthpiece has a wall thickness of from 0.2 mm to 0.8 mm, preferably from 0.3 mm to 0.7 mm, and more preferably 0.4 mm to 0.6 mm.

The mouthpiece suitably comprises a marker, the marker indicating the correct distance for insertion of the mouthpiece into the mouth of a user. The marker can be a visible marker, or it could be a somatosensory (e.g. tactile) marker.

In a further aspect of the present invention there is provided a method of operating a gas sensor device comprising providing a gas sensor as set out above, providing a suitable mouthpiece and attaching the mouthpiece to the gas sensor device. The method may comprise causing a subject to exhale into the gas sensing device through the mouthpiece. The method may comprise causing a user to insert the mouthpiece into their mouth to a distance indicated by a marker provided on the mouthpiece.

In a further aspect the present invention provides a kit comprising a gas sensor device and/or an interface as set out above with one or more mouthpieces.

In a further aspect the present invention provides a carbon monoxide or hydrogen gas sensor device comprising a replaceable mouthpiece attached thereto (e.g. via an interface), the mouthpiece comprising a tube having a cross-sectional area of 180 mm² or less, more preferably 80 mm² or less, optionally 50 mm² or less.

It is not known in the art to use a narrow-bore mouthpiece with a carbon monoxide or hydrogen gas sensor device. Such a mouthpiece provides significant advantages when used with a carbon monoxide or hydrogen gas sensor device, as will be discussed further below.

Various properties of suitable narrow-bore mouthpieces are discussed above in relation to earlier aspects of the invention. Preferably the mouthpiece has a substantially constant cross-section, e.g. a circular or elliptical cross-section. Preferably the mouthpiece is from 70 mm to 130 mm long, preferably from 80 mm to 120 mm long, and more preferably from 90 mm to 110 mm long. Suitably the mouthpiece has a wall thickness of from 0.2 mm to 0.8 mm, preferably from 0.3 mm to 0.7 mm, and more preferably 0.4 mm to 0.6 mm. The mouthpiece suitably comprises a marker, the marker indicating the correct distance for insertion of the mouthpiece into the mouth of a user.

In a further aspect there is provided a method of operating a carbon monoxide or hydrogen gas sensor device comprising providing a carbon monoxide or hydrogen gas sensor device and attaching thereto a mouthpiece comprising a tube having a cross-sectional area of 180 mm² or less, more preferably 80 mm² or less, optionally 50 mm² or less. The method can comprise inserting the mouthpiece into a user's mouth, e.g. to a distance indicated by a marker provided on the mouthpiece. The method can comprise causing a subject to exhale into the gas sensor device.

In a further aspect there is provided a kit comprising a carbon monoxide or hydrogen gas sensor device and one or more mouthpieces comprising a tube having a cross-sectional area of 180 mm² or less, more preferably 80 mm² or less, optionally 50 mm² or less.

In a further aspect the present invention provides the use of a mouthpiece having a cross-sectional area of 180 mm² or less with a carbon monoxide or hydrogen gas sensor device.

### BRIEF DESCRIPTION OF THE SEVERAL VIEWS OF THE DRAWINGS

Embodiments of the invention will now be described, by way of example only, with reference to the accompanying drawings in which:

FIG. 1 shows line drawing side and front views an interface according to the present invention;

FIG. 2 shows a cross-sectional view of the same interface as FIG. 1;

FIG. 3 shows a cross-sectional view of the second mouthpiece engaging means of the same interface;

FIG. 4 shows side and end views of a mouthpiece;

FIG. 5 shows line drawing side cross-section and isometric views of the interface with a mouthpiece attached to the second mouthpiece engaging means of the interface;

FIG. 6 shows a mouthpiece packaged prior to use; and

FIG. 7 shows cross-section and isometric views of a second embodiment of an interface according to the present invention.

### DETAILED DESCRIPTION OF THE INVENTION

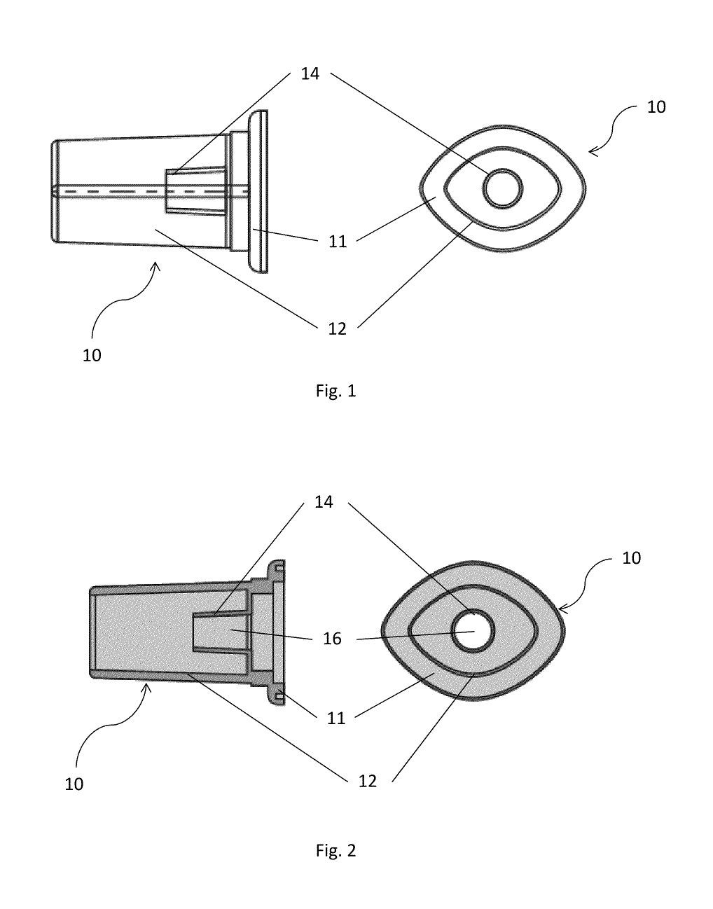

As shown in FIGS. 1 and 2, there is provided an interface 10, for a breath sensor device comprising a first mouthpiece engaging means 12 and a second mouthpiece engaging means 14. Inside the second mouthpiece engaging means there is an inlet 16 through which exhaled air passes from the lumen of the mouthpiece into the gas sensor device.

The first mouthpiece engaging means 12 comprises a substantially elliptical flange which projects from the base 11 of the interface 10. The flange tapers gently from the proximal end (i.e., the end nearest the gas sensor device) to the distal end such that the circumference of the first mouthpiece engaging means 12 decreases slightly as it extends away from the base 11. This facilitates initial attachment of a mouthpiece to the first mouthpiece engaging means, and allows for a suitably tight attachment as the mouthpiece is slid along the first mouthpiece engaging means. The first mouthpiece engaging means 12 is suitable for the attachment of a conventional mouthpiece in the form of a cardboard tube (e.g. of the type available from Bedfont Scientific Ltd, UK).

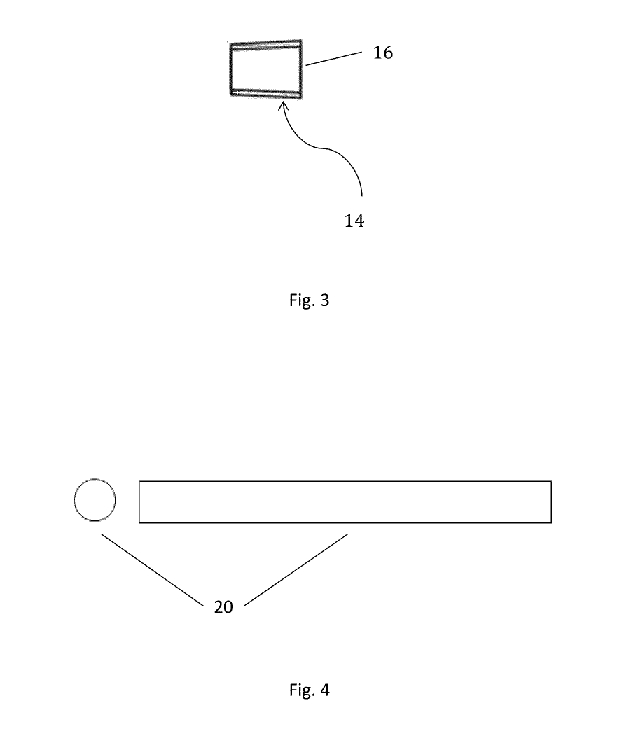

The second mouthpiece engaging means 14 comprises a substantially circular flange which extends from the base 11 of the interface 10, and which is located within the first mouthpiece engaging means 12. The second mouthpiece engaging means 14 is tapered in a similar manner to the first mouthpiece engaging means 12. The first and second mouthpiece engaging means 11,12 are concentric in the embodiment shown, though it is not necessary that they be so. FIG. 3 shows a more detailed view of the second mouthpiece engaging means, which includes an outer diameter which tapers from 10.1 mm at the proximal end to 9.9 mm at the distal end, and a constant wall thickness of 1.5 mm. The second mouthpiece engaging means 12 is suitable for the attachment of a mouthpiece having a significantly smaller diameter than the conventional mouthpieces used in respect of breath sensor devices. The specific embodiment illustrated is suited for the attachment of a mouthpiece of generally circular cross-section, and having a diameter of 10 mm. The taper allows such a mouthpiece to be easily placed over the second mouthpiece engaging means, given the reduced diameter, and allows the mouthpiece to be fitted firmly and sufficiently airtight by sliding the mouthpiece further onto the second mouthpiece engaging means 14. Typically the material of the mouthpiece will be slightly resilient, allowing the mouthpiece to stretch slightly as is slides onto the second mouthpiece engaging means until it abuts against the base 11 of the interface 10. Thus a snug and sufficiently airtight seal can be easily achieved.

The interface is connected to a breath sensor device, e.g. a CO or H₂ sensor. The base 11 can be connected to the device by a clip mechanism and/or can be retained in position by fixing means such as screws or adhesive. In the embodiment shown the interface is connected to the device using screws.

FIG. 4 shows a suitable mouthpiece 20 for attachment to the second mouthpiece engaging means 14. It is a circular cross-section tube having a substantially constant diameter of 8 mm and a length of 100 mm. The mouthpiece has a wall thickness of approximately 0.5 mm. Such a thickness has been found to provide a suitably strong tube which does not collapse when a user applies pressure to seal their lips around it, but which minimises use of material to avoid wastage and minimise cost. Suitably the tube is formed from a polymer, although other materials could be used provided they are compatible with the intended use, are non-toxic, and do adversely interact with the target gas. For example, a cardboard or paper tube could potentially be used. Suitable polymeric materials include polyurethane, polypropylene and polyethylene, but many other polymeric materials may be suitable.

The mouthpiece can comprise an antimicrobial agent. For example, the polymer may comprise an antimicrobial additive, e.g. triclosan which is a chlorinated aromatic compound with antibacterial, antifungal and antiviral properties (sold under several trade names including Microban, UltraFresh, Amicor, and BioFresh). Alternatively, silver compounds can be impregnated or combined with various materials to provide antimicrobial effects.

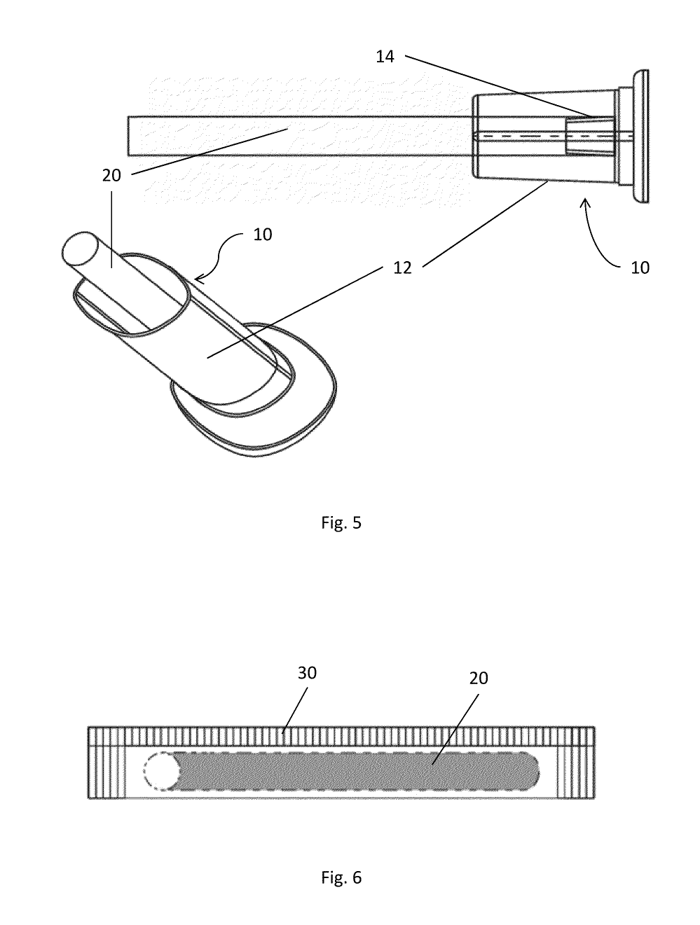

FIG. 5 shows the interface 10 with a mouthpiece 20 attached to the second mouthpiece engaging means 14.

As shown in FIG. 6 a mouthpiece 20 can be packaged in a wrapper 30, which allows the mouthpiece to be kept clean and optionally sterile prior to use. The wrapper 30 is formed from a sheet material, such as a polymer of paper. The wrapper is initially a flat rectangle, which is wrapped around the mouthpiece 20 and then the edges are sealed, e.g. using an adhesive or through thermal or ultrasonic welding; the preferred sealing method depends on, for example, the wrapper material. In FIG. 5 the sealed areas are shown with vertical hatching, and are 10 mm long at each end, and 5 mm wide down the side. The lumen in which the mouthpiece is held is approximately 120 mm long.

The present interface provides significant improvements over prior art interfaces for breath sensor devices, as will be discussed below. It can be used essentially any type of breath sensor device, but in preferred embodiments the sensor device is a carbon monoxide or hydrogen sensor device.

The ability to attach a narrow-bore mouthpiece provides several advantages. Narrow-bore mouthpieces make it easier for a user to exhale into the device at a desired flow rate. Conventional wide-bore mouthpieces provide minimal resistance to exhalation; indeed, that is an effect of their original purpose in spirometry and peak flow devices where any restriction to exhalation is undesirable. However, in breath sensor devices which detect concentrations of a constituent gas this feature is not advantageous, and indeed the present invention resides at least partially in the observation that it can be positively disadvantageous.

With a wide-bore mouthpiece it is all too easy for a subject to exhale too quickly and therefore run out of breath before a successful measurement has been taken. Even with modern sensors it still takes several seconds for a measurement to be accurately taken, and it is desirable to take the measurement during the middle of an exhalation. Where a sample is taken at the end phase of exhalation, which often occurs if a subject exhales too quickly initially, gas levels can be altered due to the comparatively high proportion of air from the residual volume of the lungs. This is obviously detrimental to the usefulness of the test because the concentration of target gas detected, though accurately measured, is not representative of typical tidal exhalation concentrations.

Furthermore, as many users are familiar with spirometry or peak flow type tests, where rapid exhalation is desirable, when confronted with a similar mouthpiece to that which is used in the test which is familiar to them, there can be a predisposition for such users to exhale rapidly. Although such a tendency can perhaps be countered by instructions being given to a subject doing the test by a clinician, this relies on the clinician giving correct instructions and, moreover, requires that a clinician is actually present, which adds to the cost of testing and may not be possible with home tests, for example. Written instructions are often not read and/or followed.

Spirometry mouthpieces are generally available in two forms, adult and paediatric. These forms are both typically circular, semi-rigid tubes, usually formed of cardboard, and the former has a diameter of approximately 30 mm and the latter of approximately 22 mm. Both of these mouthpieces are commonly used in gas sensor devices.

By using a narrow-bore mouthpiece instead, this provides a degree of resistance which tends to reduce the rate of exhalation. Providing a degree of exhalation resistance allows a subject to gauge how fast they are exhaling and adjust the rate accordingly. Where a wide mouthpiece is used and there is very little resistance to exhalation, it is extremely difficult to judge how fast one is exhaling. Furthermore, even absent a significant amount of resistance, it appears that a user is more easily able to control exhalation rate when using a narrow mouthpiece rather than a wide one. The reason for this is unclear, but perhaps is due to a user being accustomed to controlling the flow rate of exhaled air when exhaling through pursed lips, e.g. as used when whistling or when blowing normally. Additionally, because the mouthpiece is not of the type conventionally used in spirometry and peak flow testing, subjects familiar with such tests will not be predisposed to use the already similar, i.e. fast, exhalation style.

Further advantages can be provided by using a mouthpiece which is adapted to extend into the mouth beyond the teeth. For example, a mouthpiece which extends 4 cm or more into the mouth (i.e. this distance beyond the lips), can reach past the teeth of a subject into the oral cavity towards the isthmus faucium. Such a mouthpiece ensures that contamination by gases produced by dental microbes is kept to a minimum. The mouthpiece should not extend so far as to cause gagging, to impact on the throat, or to be occluded by tissues of the mouth or throat. Conveniently, a marker can be provided on a mouthpiece to provide guidance to a user as to how far the mouthpiece should be inserted into the mouth, e.g. a marker to show where a user's lips should be placed.

Using narrow-bore mouthpieces has the advantage of reducing the amount of material per mouthpiece and thus reducing costs and environmental impact. Furthermore it reduces the bulk of the packaged products considerably.

The interface described above provides the benefits described above by allowing convenient connection of a narrow-bore mouthpiece. However, it also allows a conventional wide-bore mouthpiece to be attached to the device. Thus dual utility is a distinct advantage in settings where both types of mouthpieces may be found, e.g. in medical facility where spirometry devices and associated mouthpieces are also present. A particular advantage of the interface is that even when a wide-bore mouthpiece is attached, the inlet is of restricted size compared with the diameter of the attached mouthpiece and therefore there is a degree of flow restriction provided. This therefore makes it easier for a subject to control exhalation rate.

It should be noted that the above described device and interface is a preferred embodiment of the invention, but various other embodiments within the scope of the invention are also envisaged. For example, carbon monoxide and hydrogen breath sensor devices are not known in the art in combination with a mouthpiece having a relatively small diameter. As mentioned in the introduction, CO and H₂ sensor devices are conventionally used in conjunction with a wide-bore mouthpiece, essentially as used in spirometry devices.

Modification of a CO or H₂ breath sensor device to comprise an interface adapted to engage a narrow-bore mouthpiece (e.g. having a diameter of 15 mm or less) allows the abovementioned advantages associated with narrow-bore mouthpieces to be provided to CO or H₂ sensing. This could be achieved by providing a multi mouthpiece interface as discussed above. Alternatively, in another aspect of the invention, this can be achieved by providing a single mouthpiece interface to the CO or H₂ breath sensor, the interface having a mouthpiece engaging means which is adapted to engage a narrow-bore mouthpiece.

Referring to a device comprising a dual mouthpiece interface, to use the device a user takes a narrow-bore mouthpiece and removes it from its wrapper. The mouthpiece is then pushed onto the second mouthpiece engaging means until it is securely mounted in position in the gas sensor device—this may be the point when the mouthpiece abuts against the base of the interface. The user then ensures the gas sensor device is operating and ready to receive a sample. The mouthpiece is then inserted into the mouth, preferably with the distal tip of the mouthpiece extending well past the teeth and into mouth cavity. The correct distance of insertion can be indicated by a mark provided on the mouthpiece, for example that the user aims to close their lips upon. Furthermore, the length of the mouthpiece can be selected such that over-insertion of the mouthpiece such that it touches the back of the throat or otherwise causes gagging is avoided. For example, the length of the mouthpiece can be selected such that, for a typical user, their lips will contact the interface or another part of the breath sensor device before the tip of the mouthpiece extends too far into the mouth, e.g. to contact the back of the mouth or throat. A length of 100 mm has been found to work well. Once the lips have been sealed around the mouthpiece, the user exhales into the device to provide a sample. Exhalation is continued until the device indicates that the test has been conducted.

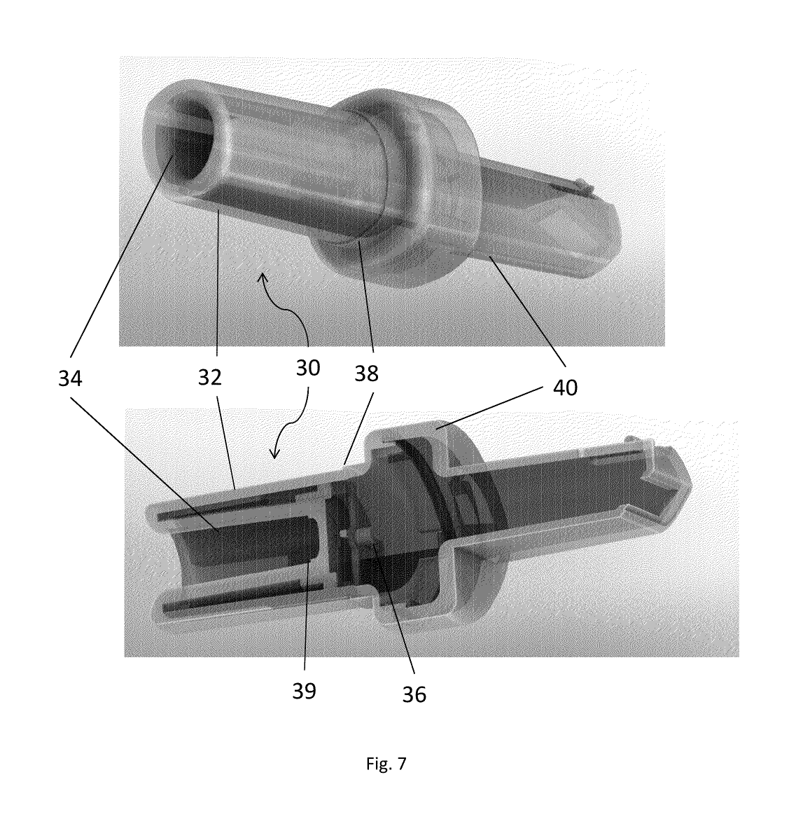

A second embodiment of the invention is shown in FIG. 7. In this embodiment the interface 30 comprises a first mouthpiece engaging means 32 and a second mouthpiece engaging means 34. Within the ambit of the second mouthpiece engaging means there is an inlet 36 through which exhaled air passes from the lumen of the mouthpiece into the gas sensor device. The interface is shown connected to a housing 40 which is part of the gas sensor device.

In this second embodiment the interface comprises a single flange, the outer surface of which defines the first mouthpiece engaging means 32, and the inner surface of which defines the second mouthpiece engaging means 34.

The first mouthpiece engaging means 32 is generally elliptical in section and tapers gently outwards in the proximal direction. Near the proximal end of the first mouthpiece engaging means there is a shallow annular shoulder 38 which projects outwards. A wide-bore mouthpiece can thus be slid onto the first mouthpiece engaging means, i.e. over the flange, the taper resulting in an increasingly tight fit as it is slid further on, until the mouthpiece abuts against shoulder 38 once it is correctly located.

The second mouthpiece engaging means 34 is generally circular in section and tapers gently inward in the proximal direction. Near the proximal end of the second mouthpiece engaging means there is a shallow annular shoulder 39 which projects outwards. A narrow-bore mouthpiece can thus be slid into the second mouthpiece engaging means, i.e. into the lumen defined by the flange, the taper resulting in an increasingly tight fit as it is slid further into the lumen, until the mouthpiece abuts against shoulder 39 once it is correctly located.

The flange thus has a wall thickness which increases in the proximal direction which provides the taper to the first and second mouthpiece engaging means. In the embodiment shown in the figures the flange has a hollow wall structure, i.e. it has an inner and outer wall with a cavity therebetween, and there is an end wall portion which joins the inner and outer wall together at the distal end, thus forming a single hollow walled flange. However, it should be noted that the wall of the single flange need not have the hollow, double-walled structure described, e.g. it could be solid. The thickness of the wall of the flange generally corresponds to the difference between the diameters of the narrow- and wide-bore mouthpieces.

This second embodiment has certain advantages over the embodiment described above. Where two separate flanges are provided there is a risk that condensation can form in the trough between the outer and inner flanges. Such condensation may not evaporate between uses of the device and thus there is a risk that droplets of condensation could run from the trough down the mouthpiece and contact a user. Furthermore, there is a risk that the trough could become unsanitary if not regularly cleaned, e.g. because the damp environment would be suitable for the growth of microbes or simply because dust and dirt could become trapped there. Any such problems could be avoided by regular cleaning and/or drying of the interface, but it would be preferably if the potential for the problem to arise were removed. The second embodiment avoids the presence of such a trough by using a single flange with a comparatively thick wall, thus allowing the single flange to provide both the first and second mouthpiece engaging means via its inner and outer surfaces. The second embodiment thus provides significant improvements in terms of avoiding cross infection and/or an unpleasant experience for a user.

Various modifications to the invention can be made without departing from the spirit of the invention. For example, the mouthpiece engaging means could comprise a groove or other recess rather than a flange. The flanges of the mouthpiece engaging means described in the preferred embodiment above are continuous elliptical or circular flanges, but discontinuous flanges could be used. Additional retention means can be provided to hold the mouthpiece in position, e.g. high friction regions on the mouthpiece engaging means or a clip mechanism.

The invention in which an exclusive property or privilege is claimed is defined as follows:

1. An interface for the attachment of a mouthpiece to a gas sensor device, the interface comprising a first mouthpiece engaging means for engaging a first type of mouthpiece, and a second mouthpiece engaging means for engaging a second type of mouthpiece, the interface comprising an inlet positioned such that in use it is in fluid communication with the lumen of a mouthpiece mounted on either the first or the second mouthpiece engaging means.

2. The interface of claim 1, wherein the first mouthpiece engaging means is adapted to engage a larger mouthpiece than the second mouthpiece engaging means.

3. The interface of claim 1, wherein the first and/or second mouthpiece engaging means comprises a flange adapted to engage with the mouthpiece.

4. The interface of claim 1, wherein the first and/or second mouthpiece engaging means comprises a recess adapted to engage with the mouthpiece.

5. The interface of claim 3, wherein the first and second mouthpiece engaging means each comprises a flange.

6. The interface of claim 1, wherein the first and second mouthpiece engaging means are defined by a flange, the outside of the flange defining the first mouthpiece engaging means and the inside of the flange defining the second mouthpiece engaging means.

7. The interface of claim 1, wherein the first and/or second mouthpiece engaging means defines a circle or ellipse.

8. The interface of claim 7, wherein both the first and second mouthpiece engaging means define a circle or ellipse.

9. The interface of claim 8, wherein the first mouthpiece engaging means defines an ellipse and the second mouthpiece engaging means defines a circle.

10. The interface of claim 1, wherein the first and second mouthpiece engaging means are concentric.

11. The interface of claim 1, wherein the second mouthpiece is adapted to engage a mouthpiece having a lumen with a cross-sectional area of approximately 180 mm² or less.

12. The interface of claim 1, wherein the first mouthpiece is adapted to engage a mouthpiece cross-sectional area of approximately 300 mm² or more.

13. The interface of claim 1, wherein the inlet is positioned within the ambit of the second mouthpiece engaging means.

14. A gas sensor device having an interface comprising a first mouthpiece engaging means for engaging a first type of mouthpiece, and a second mouthpiece engaging means for engaging a second type of mouthpiece, and the interface comprising an inlet positioned such that in use it is in fluid communication with the lumen of a mouthpiece mounted on either the first or the second mouthpiece engaging means.

15. The gas sensor device according to claim 14, further comprising a mouthpiece attached to the first or second mouthpiece engaging means.

16. The gas sensor device according to claim 15, wherein the mouthpiece is attached to the second mouthpiece engaging means and has a substantially circular cross-section and a diameter of approximately 10 mm or less.

17. The gas sensor device according to claim 15, wherein the mouthpiece is from approximately 70 mm to approximately 130 mm long.

18. The gas sensor device according to claim 15, wherein the mouthpiece comprises a marker, the marker indicating the correct distance for insertion of the mouthpiece into the mouth of a user.

19. The gas sensor device according to claim 19, wherein the mouthpiece has a lumen with a cross-sectional area of approximately 180 mm² or less.

20. A method of operating a gas sensor device, comprising the steps of:
Providing a gas sensor device having an interface comprising a first mouthpiece engaging means for engaging a first type of mouthpiece, and a second mouthpiece engaging means for engaging a second type of mouthpiece, and the interface comprising an inlet positioned such that in use it is in fluid communication with the lumen of a mouthpiece mounted on either the first or the second mouthpiece engaging means;
providing a suitable mouthpiece; and
attaching the mouthpiece to the gas sensor device.

21. The method of claim 20, wherein the mouthpiece has a cross-sectional area of approximately 180 mm² or less.

22. The method of claim 20, further comprising the step of causing a subject to be tested to exhale into the gas sensing device through the mouthpiece.

23. The method of claim 22, wherein the mouthpiece comprises a marker, the marker indicating the correct distance for insertion of the mouthpiece into the mouth of a user; and wherein further the step of causing a subject to be tested to exhale into the gas sensing device through the mouthpiece includes causing the subject to be tested to insert the mouthpiece into their mouth to a distance indicated by a marker provided on the mouthpiece.

24. A carbon monoxide or hydrogen gas sensor device, comprising a replaceable mouthpiece attached thereto via an interface, the mouthpiece comprising a tube having a cross-sectional area of approximately 180 mm² or less.

25. The carbon monoxide or hydrogen gas sensor device of claim 24, wherein the mouthpiece has a substantially constant cross-section.

26. The carbon monoxide or hydrogen gas sensor device of claim 24, wherein the mouthpiece has a circular or elliptical cross-section.

27. The carbon monoxide or hydrogen gas sensor device of claim 24, wherein the mouthpiece is from approximately 70 mm to approximately 130 mm long.

28. The carbon monoxide or hydrogen gas sensor device of claim 24, wherein the mouthpiece has a wall thickness of from approximately 0.2 mm to approximately 0.8 mm.

29. The carbon monoxide or hydrogen gas sensor device of claim 24, wherein the mouthpiece comprises a marker, the marker indicating the correct distance for insertion of the mouthpiece into the mouth of a user.

30. A method of operating a gas sensor device, comprising: providing a carbon monoxide or hydrogen gas sensor device and attaching thereto a mouthpiece having a cross-sectional area of approximately 180 mm² or less.

31. The method of claim 42, further comprising the step of causing a subject to be tested to exhale into the gas sensor device through the mouthpiece.

32. The method of claim 31, wherein the mouthpiece comprises a marker, the marker indicating the correct distance for insertion of the mouthpiece into the mouth of a user; and wherein further the step of causing a subject to be tested to exhale into the gas sensing device through the mouthpiece includes causing the subject to be tested to insert the mouthpiece into their mouth to a distance indicated by a marker provided on the mouthpiece.

33. A kit, comprising:
An interface for the attachment of a mouthpiece to a gas sensor device, the interface comprising a first mouthpiece engaging means for engaging a first type of mouthpiece, and a second mouthpiece engaging means for engaging a second type of mouthpiece, the interface comprising an inlet positioned such that in use it is in fluid communication with the lumen of a mouthpiece mounted on either the first or the second mouthpiece engaging means; and
one or more mouthpieces mountable on one or more of the first and second mouthpiece engaging means.

34. A kit, comprising:
A gas sensor device having an interface comprising a first mouthpiece engaging means for engaging a first type of mouthpiece, and a second mouthpiece engaging means for engaging a second type of mouthpiece, and the interface comprising an inlet positioned such that in use it is in fluid communication with the lumen of a mouthpiece mounted on either the first or the second mouthpiece engaging means; and
one or more mouthpieces mountable on one or more of the first and second mouthpiece engaging means.
